(19) United States
(12) Patent Application Publication
(10) Pub. No.: US 2009/0299447 A1
(43) Pub. Date: Dec. 3, 2009

(54) DEPLOYABLE EPICARDIAL ELECTRODE AND SENSOR ARRAY

(76) Inventors: Marc Jensen, Los Gatos, CA (US); Olivier Colliou, Los Gatos, CA (US); Benedict Costello, Berkeley, CA (US); Mark Zdeblick, Portola Valley, CA (US); Todd Thompson, San Jose, CA (US); George Savage, Portola Valley, CA (US); Andrew Thompson, Portola Valley, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/917,992

(22) PCT Filed: Jun. 30, 2006
(86) PCT No.: PCT/US06/25648

§ 371 Date: Jul. 8, 2009

(51) Int. Cl. A61N 1/05

(52) U.S. Cl. 607/130

(57) ABSTRACT

Minimally invasive deployable epicardial array devices are provided. The devices include deployable platform comprising two or more effectors, such as sensors and actuators, where the devices are configured to be deployed at an epicardial location via a minimally invasive, e.g., sub-xiphoid approach. In embodiments of the present invention, at least one area of the electrode patch is an electrical control area that comprises a series of effectors, e.g., sensors and/or electrodes. Other embodiments provide localized physical constraint and dynamic mechanical stimulation of the heart to effectuate physical and biological responses. Still other embodiments provide both of these functions. Also provided are methods of using the devices, as well as systems and kits that include the devices.

### CROSS-REFERENCE TO RELATED APPLICATIONS

Pursuant to 35 U.S.C. § 119 (e), this application claims priority to the filing dates of: U.S. Provisional Patent Application Ser. No. 60/696,317 filed Jul. 1, 2005; U.S. Provisional Patent Application Ser. No. 60/706,641 filed Aug. 8, 2005; and U.S. Provisional Patent Application Ser. No. 60/806,309 filed Jun. 30, 2006; the disclosures of which are herein incorporated by reference.

### BACKGROUND

The present Invention relates generally to sensors and actuators for use in medical methods, apparatuses and systems. More specifically, the invention relates to methods, apparatuses and systems for optimizing cardiac resynchronization intervention, arrhythmia management, ischemia ejection, coronary artery disease management, and heart failure management.

Epicardial electrode leads are devices which are placed on the epicardial surface of the heart and used to pace and sense. Unfortunately, the designs are very limited and are either placed via open chest, a mini-thoracotomy, or a thoracoscopic approach. While the latter two approaches can be considered minimally invasive, they still require multiple, relatively large, incisions and rigid delivery tools. Furthermore, the current epicardial electrode leads are limited to one or two electrodes.

Stress reduction harnesses are devices which surround and restrain the heart. The term “cardiac harness” as used herein is a broad term that refers to a device fit onto a patient's heart to apply a compressive force on the heart during at least a portion of the cardiac cycle. Other devices that are intended to be fit onto a heart and are referred to in the art as “girdles,” “socks,” “jackets,” or the like are included within the meaning of “cardiac harness.” A recent example of such a device is described in United States Patent Application publication 20040143154 filed on Jul. 22, 2004 to Lau et al. These devices can go beyond physical constraint to provide electrical stimulation and sensing. See United States Patent Application publication 20050102011 published on May 12, 2005 to Lau et al.

These devices are made to restrict the heart physically. Because they must completely surround the heart to be effective, they can be difficult to install when there are adhesions connecting the heart to surrounding tissues. Additionally, installing these devices is typically a seriously invasive procedure.

Most recently, Lau et al have disclosed a self-anchoring cardiac harness which can limit the necessity for suturing full heart socking devices. See United States Patent Application publication 20050054892 published on Mar. 10, 2005.

Deployable heart stents are well known in the art. They enjoy a substantial advantage over heart socks in that they can be deployed in a minimally invasive manner, with limited tissue trauma. An example is found in United States Patent Application publication 20020040236 published on Apr. 4, 2002 by Lau et al, which describes such procedures.

It would be an important clinical advancement if some of the advantages of heart socks could be accomplished using very minimally invasive procedures and devices, and their effects could be provided in a more physically strategic approach to a treated organ or body region, including optimized placement and lower installation trauma.

### SUMMARY OF THE INVENTION

The new and novel concept of multiplexing pacing and sensing signals developed by the present inventors which has special epicardial applications has provided new configurations of electrodes provided by the deployable arrays of the present invention. In embodiments of the present invention, at least one area of the electrode patch or net is an electrical control area that comprises a series of sensors and/or leads. Others provide localized physical constraint and dynamic mechanical stimulation to effectuate physical and biological responses. Still other embodiments provide both of these functions. The deployable devices are configured to be delivered in a minimally invasive manner. The delivery system to place the described epicardial devices is flexible and minimally invasive.

Special applications of the present invention are in the area of cardiac rhythm management. Devices used in cardiac resynchronization can be deployed epicardially by the present inventive deployable patches or nets to combine several treatment modalities that are particularly beneficial to patients suffering from congestive heart failure. The inventive deployable patch or net can provide a selective compressive force on the heart when deployed with attachment or other anchoring means. This serves to relieve wall stress, and improve cardiac function. The defibrillating and pacing/sensing electrodes associated with the inventive deployable electrode patches or nets along with suitable control devices, such as implantable cardioverter defibrillators (ICDs) and artificial pacemakers, provide numerous treatment options to correct for any number of maladies associated with congestive heart failure.

Cardiac rhythm devices deployed on the inventive patch or net can provide electrical pacing stimulation to one or more of the heart chambers to improve the coordination of atrial and/or ventricular contractions, that is resynchronization therapy. Cardiac resynchronization therapy is pacing stimulation applied to one or more heart chambers, typically the ventricles, in a manner that restores or maintains synchronized bilateral contractions of the atria and/or ventricles thereby improving pumping efficiency.

Resynchronization pacing may involve pacing both ventricles in accordance with a synchronized pacing mode. For example, pacing at more than one site (multi-site pacing) at various sites on the epicardial surface of the heart to desynchronize the contraction sequence of a ventricle (or ventricles) may be therapeutic in patients with hypertrophic obstructive cardiomyopathy, where creating asynchronous contractions with multi-site pacing reduces the abnormal hyper-contractile function of the ventricle. Further, resynchronization therapy may be implemented by adding synchronized pacing to the bradycardia pacing mode where paces are delivered to one or more synchronized pacing sites in a defined time relation to one or more sensing and pacing events.

An example of synchronized chamber-only pacing is left ventricle only synchronized pacing where the rate in synchronized chambers are the right and left ventricles respectively. Left-ventricle-only pacing may be advantageous where the conduction velocities within the ventricles are such that pacing only the left ventricle results in a more coordinated contraction by the ventricles than by conventional right ventricle pacing or by ventricular pacing.

Further, synchronized pacing may be applied to multiple sites of a single chamber, such as the left ventricle, the right ventricle, or both ventricles. Pacemakers which can be, in some cases, associated with the present invention are typically implanted subcutaneously on a patient's chest and have leads threaded to the pacing/electrodes in order to connect the pacemaker to the electrodes for sensing and pacing. The pacemakers sense intrinsic cardiac electrical activity through the electrodes disposed on the surface of the heart. Pacemakers are well known in the art and any commercially available pacemaker or combination defibrillator/pacemaker can be used in accordance with the present invention.

### BRIEF SUMMARY OF THE DRAWINGS

FIG. 1A is a depiction of an epicardial multielectrode patch lead employing a multiplexing system and a pre-shaped spiral configuration which allows it to be deployed minimally invasively via a steerable catheter. A bioabsorbable clip is used to temporarily fix the epicardial device in place. Also shown in FIGS. 1B to 1D are pre-shaped accordion and star or finger configurations of a deployable platform of embodiments of the invention.

FIG. 2 is partial sectional view of an epicardial multielectrode lead with a flattened cross section and an electrode exposed only on one side of the lead. This design allows the lead to track more easily around the heart with electrodes preferentially oriented to only contact the heart.

FIG. 3 is a depiction of an RF ablating dissection tool which is used to tunnel through adhesions in the pericardial space for placement of an epicardial electrode lead of the invention.

FIG. 4 is a depiction of a steerable rail-guided stapler used for fixation of the epicardial electrode lead to the heart.

FIG. 5 is a depiction of a multielectrode epicardial heart basket which uses one-wire technology as described in published United States Patent Application Publication Number 2006/0058588 to select, pace, and sense from any combination of electrodes.

FIG. 6A is a depiction of an epicardial multielectrode expandable net employing a multiplexing system which can be placed via a catheter and then spread-sail deployed at a desired epicardial location. FIG. 6B is a depiction of a balloon element that can be used to stabilize the epicardial multielectrode net against the heart during minimally invasive fixation with sutures, staples, or electrically active microhooks.

FIG. 7A is a depiction of an epicardial multielectrode net lead employing a pre-shaped loop configuration. FIG. 7B is a second embodiment with a pre-shaped rhombus configuration is also shown.

FIG. 8 is a depiction of an epicardial multielectrode lead which is sutured into the heart wall.

FIG. 9 is a depiction of an epicardial multielectrode lead which is placed in the heart by puncturing the myocardium. A microchip embedded at the electrode location is used to activate any combination of electrodes.

FIG. 10 is a depiction of a steerable flexible suction and delivery device used to stabilize the heart, deliver and attach an epicardial device.

FIG. 11 is a depiction of a suction device used to create a vacuum in the pericardial cavity in order to temporarily stabilize an epicardial device against the heart during fixation.

FIG. 12A is a depiction of a balloon device used to temporarily stabilize an epicardial device against the heart during fixation with staples, sutures, or other devices. Also shown in FIG. 12B is a spring clip device with the same purpose.

FIG. 13 is a depiction of a conductive (e.g. Pt microspheres or carbon fibers in cyanoacrylate) or non-conductive adhesive which is used for fixation of the epicardial multielectrode leads.

FIGS. 14A to 14E provide depictions of several embodiments of epicardial leads using pneumatic, bevel gears, universal joint, and cable winding mechanisms to rotate the active fixation helix electrode.

FIGS. 15A and 15B are depictions of wireless epicardial devices which are powered and controlled by a subdermally implanted device (e.g. RF coil for power and data transmission) and pacemaker.

FIG. 16 is a depiction of a pericardial pressure sensing device which includes a differential pressure sensor.

FIG. 17A is a depiction of an epicardial partial mechanical constraint device that is comprised of a minimally invasively delivered mesh patch which is locally fixated to the heart using multiple sutures, staples, or microhooks. Also shown in FIG. 17B is an epicardial multi-balloon multi-electrode lead employing a pre-shaped spiral. A bioabsorbable clip is used to temporarily fix the epicardial device in place. An implantable pacemaker and pump is used to pace and sense from the electrodes and inflate and deflate the balloons for mechanical stimulation.

FIG. 18 illustrates a sinusoidal shaped lead with uniform bends along the lead's length.

FIG. 19a illustrates a sinusoidal shaped lead with decreasing bends 9 in the distal direction, and electrodes 7 located at the apex of the bends of the lead.

FIG. 19b illustrates a sinusoidal shaped lead with uniform bends along its length, and electrodes located off of the apex of the bends. FIG. 29c illustrates a spiral shaped lead.

FIG. 20a is an illustration of the multi-dimensional array lead with the lead positioned in the plane of the epicardial surface. FIG. 20b is an illustration of the multi-dimensional array lead with the lead positioned in a plane perpendicular to the epicardial surface, so that the lead is wedged between the epicardium and the pericardial sac.

FIG. 21a illustrates the multi-dimensional array lead with a chisel lead tip.

FIG. 21b illustrates the multi-dimensional array lead with a lumen through which a guide wire may be inserted.

FIG. 22a is a cross-sectional view of an embodiment of a lead according to the invention with a circular cross-section. FIG. 22b is a cross-sectional view of the an embodiment of a lead according to the invention with a rectangular cross-section. FIG. 22c is an electrode configured so that the anode is placed adjacent to the cathode. FIG. 22d is an electrode configured with the anode positioned inside of the cathode.

FIG. 23 is an illustration of a lead placed inside of a vein which then exits the vein.

FIG. 24a is a sinusoidal shaped lead placed inside a vein on the outside of the heart. FIG. 24b is an illustration of a sinusoidal shaped lead which exits a vein and traverses the space between the epicardial surface and the pericardium.

FIG. 24c is an illustration of a sinusoidal shaped lead which enters the surface between the epicardium and the pericardium through a sub-apex approach.

FIG. 25a is an illustration of a sinusoidal shaped lead with decreasing bends in the distal direction, which traverses the space between the epicardial surface and the pericardium. FIG. 25b is an illustration of a lead which enters the surface between the epicardium and the pericardium through a sub-apex approach with a “U” shaped lead that turns back on itself in the upper region of the ventricle.

### DETAILED DESCRIPTION OF THE INVENTION

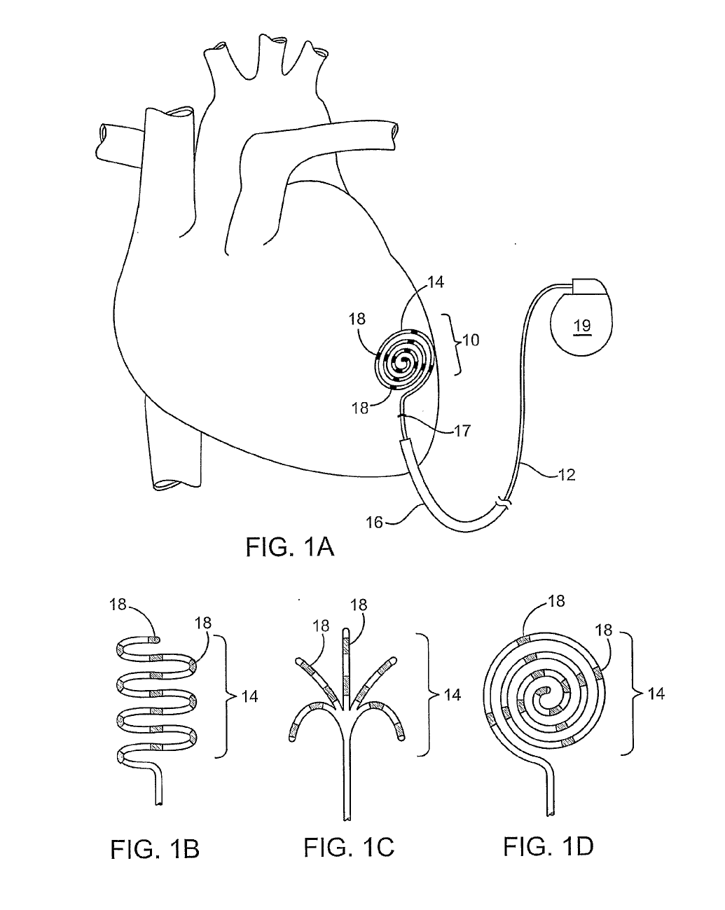

The new and novel concept of multiplexing pacing and sensing signals developed by the present inventors has special epicardial applications made possible by novel configurations of electrodes made available for the first time by the deployable arrays of the present invention shown in FIGS. 1-17B. In embodiments of the present invention, at least one area of the electrode patch is an electrical control area that comprises a series of effectors, e.g., sensors and/or electrodes. Other embodiments provide localized physical constraint and dynamic mechanical stimulation of the heart to effectuate physical and biological responses. Still other embodiments provide both of these functions.

Embodiments of the invention are facilitated by use of multiplex leads, in which two or more effectors are present on a multiplex electrical lead structure. A variety of multiplex lead formats are known in the art and may readily be adapted for use in the present devices. See e.g., U.S. Pat. Nos. 5,593,430; 5,999,848; 6,418,348; 6,421,567 and 6,473,653; the disclosures of which are herein incorporated by reference. Of particular interest are multiplex leads as disclosed in published U.S. Patent application no. 2004/0193021; the disclosure of which is herein incorporated by reference.

An important technology which facilitates direct, practical application of the present innovation is a one-wire approach to activation and control of the effectors, e.g., sensors and actuators of the present invention. This innovation is provided by one of the present inventors in United States Patent Application Publication Number 2006/0058588 (the disclosure of which configuration is herein incorporated by reference) which describes a one wire multiplex lead, in which each effector or satellite is coupled to a single wire and a second conduction path is established between each satellite and a controller, e.g., ICD or pacemaker can, using the body as a conduction path.

Additionally, a circuitry innovation with orders of magnitude lower powered consumption and uniquely miniaturized form factor is also very suited to implementation in the present invention. This circuitry design, by one of the present inventors, is described in United States Patent Application Publication Number 2006/0058588, the disclosures of which circuit is herein incorporated by reference.

The innovative deployable electrode patch or net point provides a physical epicardial platform for a variety of different types of actuators, such as MEMS sensors and actuating electrodes. These “deployable patches or nets” have many of the qualities of deployable partial heart socks, with various attachment and positioning means. However, they serve as a very deployable, strategic platform. The platform can perform as an electrode patch or net, or provide selective physical constraint on the heart. Multiple functions of these sorts are available in several inventive embodiments.

Much less invasive than prior art heart socks, the inventive deployable electrode patches or nets can be placed via a sub-xiphoid approach and then slipped through tight or scarred areas surrounding the heart before being spread-sailed deployed. Additionally, by deploying in a substantially smaller area, these devices are much less traumatic to the patent. As the devices can be delivered by a sub-xiphoid approach, during delivery they can be introduced through an opening having a diameter of about 20 mm or less, such as about 10 mm or less, including about 5 mm or less.

The multiplexed system of some of the present inventors is particularly suitable for use with the present deployable electrode patch or net. This is described in part in published United States Patent Application Publication Nos. 20040193021; 20040220637; 20040254483; 20040215049; 20060058588 and International application serial no. PCT/US2005/046811; the disclosures of which are herein incorporated by reference. Another related patent application is published as WO 2006/042039, the disclosure of which and priority applications thereof is herein incorporated into the present application by reference in their entirety.

The term “effector” is generally used herein to refer to sensors, activators, sensor/activators, actuators (e.g., electromechanical or electrical actuators) or any other device that may be used to perform a desired function. In some embodiments, for example, effectors include a transducer and a processor (e.g., in the form of an integrated circuit (digital or analog). As such, embodiments of the invention include ones where the effector comprises an integrated circuit. The term “integrated circuit” (IC) is used herein to refer to a tiny complex of electronic components and their connections that is produced in or on a small slice of material, i.e., chip, such as a silicon chip. In certain embodiments, the IC is an IC as described in PCT Patent Application Serial No. PCT/US2005/031559 titled “Methods And Apparatus For Tissue Activation And Monitoring” filed on Sep. 1, 2005, the disclosure of which is herein incorporated by reference.

The effectors of the deployable platforms may be intended for collecting data, such as but not limited to pressure data, volume data, dimension data, temperature data, oxygen or carbon dioxide concentration data, hematocrit data, electrical conductivity data, electrical potential data, pH data, chemical data, blood flow rate data, thermal conductivity data, optical property data, cross-sectional area data, viscosity data, radiation data and the like. As such, the effectors may be sensors, e.g., temperature sensors, accelerometers, ultrasound transmitters or receivers, voltage sensors, potential sensors, current sensors, etc. Alternatively, the effectors may be intended for actuation or intervention, such as providing an electrical current or voltage, setting an electrical potential, heating a substance or area, inducing a pressure change, releasing or capturing a material or substance, emitting light, emitting sonic or ultrasound energy, emitting radiation and the like.

Effectors of interest include, but are not limited to, those effectors described in the following applications by at least some of the inventors of the present application: U.S. patent application Ser. No. 10/734,490 published as 20040193021 titled: “Method And System For Monitoring And Treating Hemodynamic Parameters”; U.S. patent application Ser. No. 11/219,305 published as 20060058588 titled: “Methods And Apparatus For Tissue Activation And Monitoring”; International Application No. PCT/US2005/046815 titled: “Implantable Addressable Segmented Electrodes”; U.S. patent application Ser. No. 11/324,196 titled “Implantable Accelerometer-Based Cardiac Wall Position Detector”; U.S. patent application Ser. No. 10/764,429, entitled “Method and Apparatus for Enhancing Cardiac Pacing,” U.S. patent application Ser. No. 10/764,127, entitled “Methods and Systems for Measuring Cardiac Parameters,” U.S. patent application Ser. No. 10/764,125, entitled “Method and System for Remote Hemodynamic Monitoring”; International Application No. PCT/US2005/046815 titled: “Implantable Hermetically Sealed Structures”; U.S. application Ser. No. 11/368,259 titled: “Fiberoptic Tissue Motion Sensor”; International Application No. PCT/US2004/041430 titled: “Implantable Pressure Sensors”; U.S. patent application Ser. No. 11/249,152 entitled “Implantable Doppler Tomography System,” and claiming priority to: U.S. Provisional Patent Application No. 60/617,618; International Application Serial No. PCT/US05/39535 titled “Cardiac Motion Characterization by Strain Gauge”. These applications are incorporated in their entirety by reference herein.

In certain embodiments, one or more of the effectors is a segmented electrode structure made up of two or more electrodes positioned close to each other, where the electrodes can be individually activated. In certain embodiments, the segmented electrodes include at least one cathode and at least one anode from which highly localized stimulatory energy may be produced. The electrode components of each segmented electrode can be individually activated. In certain embodiments, the segmented electrodes include an integrated circuit electrically coupled to two or more electrodes, where each electrode can be individually activated.

Aspects of the invention include electrodes that are segmented, e.g., to provide better current distribution in the tissue/organ to be stimulated. In such embodiments, the segmented electrodes are able to pace and sense independently with the use of an integrated circuit (IC) in the lead, such as a multiplexing circuit, e.g., as disclosed in PCT Application No. PCT/US2005/031559 titled “Methods and Apparatus for Tissue Activation and Monitoring” and filed on Sep. 1, 2005; the disclosure of which is herein incorporated by reference. The IC allows each electrode to be addressed individually, such that each may be activated individually, or in combinations with other electrodes on the medical device. In addition, they can be used to pace in new and novel combinations with the aid of the multiplexing circuits on the IC. Of interest in certain embodiments are the electrode structures disclosed in provisional U.S. Patent Application Ser. No. 60/806,309 titled “Shaped Cardiac Lead with Multi-Dimensional Electrode Array,” having an attorney docket no. PRTS-051PRV and filed on Jun. 30, 2006.

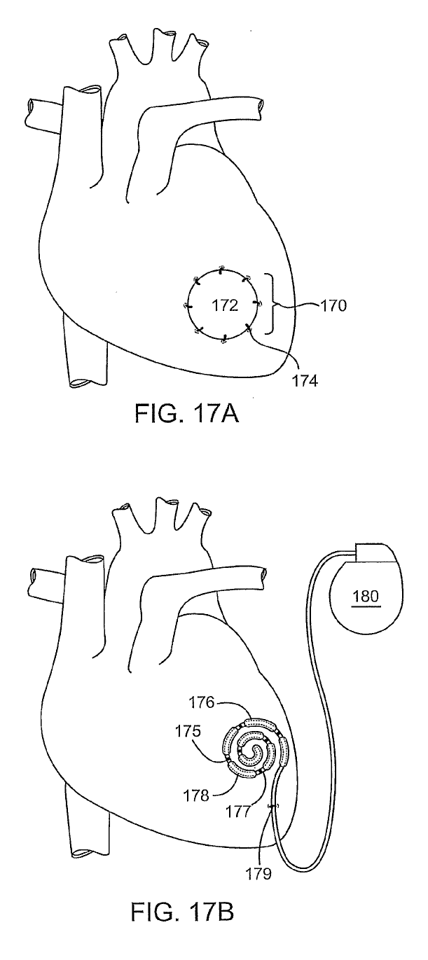

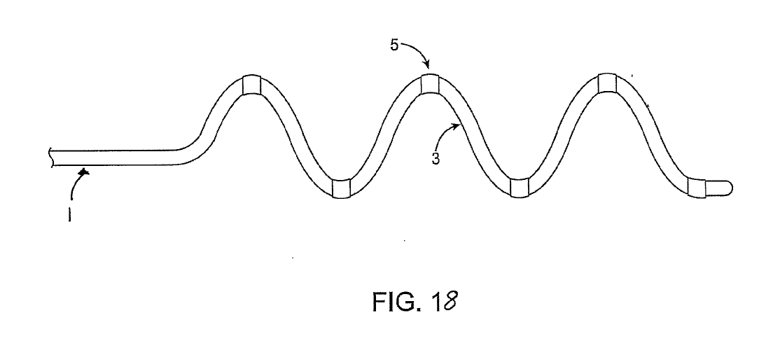

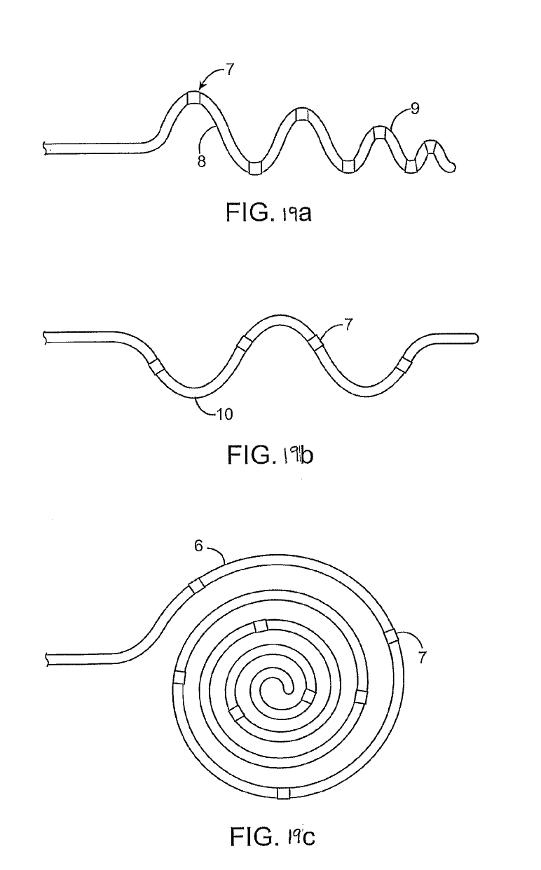

In yet other embodiments, the effectors may be mechanical actuators or stimulators, which in some way impart a mechanical stimulus to tissue that is contacted by the effector. Examples of such effectors include those described below in connection with the embodiments of FIGS. 17A and 17B, which can provide mechanical stimulation of the heart tissue to cause a cardiac biological response. It has been shown in the literature that cardiac cells are highly sensitive to changes in their mechanical environment. Applying mechanical stimulation in the form of strain or hydrostatic pressure to cardiac cells causes a biological response such as increased or decreased expression of various proteins. This cell level response translates into a cardiac tissue level response and subsequently a cardiac performance response. Mechanical stimulation of a selected region of the heart wall can be achieved, as shown in FIGS. 17A and 17B, with a passive partial constraint device in the form of a patch or net or a dynamic localized mechanical stimulation of the heart. The latter implantable epicardial device is comprised of a series of balloons which can be inflated and deflated by a pacemaker-pump device. The inflation and deflation provides dynamic mechanical stimulation to the heart by deforming the cardiac tissue and cells within the vicinity of the balloons. The biological responses induced by this mechanical stimulation may have beneficial effects on the cardiac performance and health of the patient.

As discussed above, the deployable platforms include two or more effectors, such that they include plurality of effectors. By plurality is meant 2 or more, including about 5 or more, such as about 10 or more, where the number in the plurality can be as great as about 16 or more, about 24 or more, and in certain embodiments ranges from about 1 to about 500, such as from about 5 to about 300, including from about 10 to about 100.

As indicated above, the devices include a deployable platform of one or more effectors. In certain embodiments, the distally located or positioned platform is one that can be reversibly configured from a first format or configuration to a second format or configuration. As such, the spatial arrangement of the plurality of effector elements can be reversibly changed from a first pattern to a second pattern.

In certain embodiments, the second configuration is distinguished from the first configuration by having a cross-sectional dimension, e.g., length or width, that is greater than the corresponding cross-sectional dimension of the platform in the first configuration, e.g., by about 2 to about 20 times or more. In certain embodiments, the platform is reversibly deployable. By “reversibly” is meant that the platform can be changed from the first to second configuration and then back to the first configuration as desired, e.g., as commanded by the operator of the device. As such, the platform can be readily reconfigured between the first and second configurations as desired.

The platform of certain embodiments is further characterized in that the first configuration provides for a distal end outer diameter of the device that is shorter than the distal end outer diameter of the device when the platform are present in the second configuration. The magnitude of difference in length of the outer diameter between the first and second configurations in certain embodiments is at about 2-fold or more, such as at about 3, 4, 5 fold or more. The outer diameter in the first configuration in certain embodiments ranges from 1 to about 15 Fr, including from about 1 to about 12 Fr. The shorter outer diameter in the first configuration provides for a “low catheter profile” during introduction of the distal end of the platform to the target epicardial site.

In certain embodiments, the device is configured so the platform, upon deployment is configured to mate with an epicardial region or area of the heart. Where desired, attachment elements, both temporary and permanent, may be employed to provide for immobilization of the deployed platform at the desired epicardial location.

Use of the device may include visualization of data obtained with the devices. Some of the present inventors have developed a variety of display and software tools to coordinate multiple sources of sensor information which will be gathered by use of the inventive deployable electrode patch or net. Examples of these can be seen in international PCT application serial no. PCT/US2006/012246; the disclosure of which application, as well as the priority applications thereof are incorporated in their entirety by reference herein.

The inventive leads of certain embodiments provide several advantages over prior leads, because the inventive lead deploys to form a multi-dimensional effector, e.g., electrode, array in which the electrodes can be individually activated. The multi-dimensional electrode array creates several unprecedented clinical opportunities. The ability to provide stimulation through a multi-dimensional electrode array alleviates the often traumatic problem of having to reposition a lead when the lead electrodes are in improper positions because the lead either does not provide adequate stimulation or the lead stimulates inappropriate organs such as the Phrenic nerve. With a multi-dimensional electrode array a doctor of ordinary skill will be able to focus stimulation to the optimal pacing areas, and away from problematic pacing areas by activating or deactivating certain electrodes in the array, without having to reposition the lead.

Further, the implantation of the inventive lead of certain embodiments is non-invasive, because the lead is delivered in a straight configuration with the use of guiding catheters or other delivery tools. The lead then spreads to assume a tortuous configuration when the delivery tools are exited. Several electrodes are disposed along the lead length. With the lead in a tortuous configuration, the electrodes spread out to form a multi-dimensional electrode array which covers a larger surface area or space than a straight lead or a single electrode lead.

The inventive lead has the further advantage that placement of the lead is independent of vein location, unlike leads that are used strictly in the vein anatomy, because this lead can be implanted on the surface of an organ, such as on the epicardial surface. Positioning a lead independent of the vein anatomy makes more areas of the heart available for pacing or positional measurement.

The shape of the lead can be varied depending on the application of the lead. For example, the lead can be configured as a spiral to create a patch-like configuration which will be placed on the surface of a heart organ, such as the epicardial surface. This configuration will create a multi-dimensional array about a circular region. Alternatively, the lead can be configured as a sinusoidal shape, which can be either placed on a surface of a heart organ, such as the epicardial surface, or be wedged between two surfaces such as in a vein or artery, or between the epicardium and the pericardial sac. In both cases, the configuration forms a multi-dimensional electrode array.

The electrodes of the lead can be configured to be exposed all the way around the lead body. Alternatively, the electrodes can be configured to be exposed on only a portion of the circumference of the lead body. For example, electrodes can be exposed on only one side of the lead to produce a more focused signal from the electrode, providing further control to isolate the targeted tissue for stimulation.

The electrodes in the multi-dimensional array lead can either be pacing electrodes or electrodes used for positional measurement with a cardiac resynchronization system, e.g., the SyncAssist™ system (Proteus Biomedical, Redwood City, Calif.). Similarly, in some embodiments of the present invention, non-electrode sensors can be positioned similarly to the electrode herein. By example, pressure sensors, blood velocity sensors, pH sensors, and other sensors may be so employed. As such, the examples herein may be understood to include such devices where electrodes are mentioned.

The advantage of using the multi-dimensional array lead for positional sensing is that the multi-dimensional electrode array can be placed on the epicardial surface to trace the motion of the epicardial surface over a large surface area. Further, the multi-dimensional array lead has an advantage for positional measurement because the placement of electrodes in the multi-dimensional array lead is independent of vein location, unlike leads that are used strictly in the vein anatomy.

Further, the cross sectional profile of the lead may be varied to achieve desired properties in the lead. For example, the lead may be made with a circular cross section profile to have equal bending stiffness in all directions. Alternatively, the lead can be made with a rectangular cross sectional profile to have less bending stiffness in the direction perpendicular to the plane of the surface where the lead is deployed, allowing it to bend easily around the surface, and more bending stiffness in the direction along this surface, so that the lead will maintain its desired shape in the plane of the surface.

### Materials

It is to be understood that several embodiments of deployable electrode patches or nets can be constructed and that such embodiments may have varying configurations, sizes, flexibilities, etc. The inventive patches or nets may be constructed of many suitable materials including various metals, fabrics, plastics and braided filaments. Suitable materials also include superelastic materials and materials that exhibit shape memory. For example, a preferred embodiment is constructed of Nitinol. Shape memory polymers can also be employed. Such shape memory polymers can include shape memory polyurethanes or other polymers such as those containing oligo (∈-caprolactone) dimethacrylate and/or poly(∈-caprolactone), and the like.

The inventive electrode patches or nets can be advantageously configured to break potential electrical continuity between sensors or actuators. The size of the nonconductive areas can be adjusted appropriately. It is to be understood that several types of polymer materials can acceptably be used, whether in sheet, knit, mesh or other form, to form a non-conductive area of the patch or net. For example, any medical grade polymer can be acceptable, including, for example, polyethylene, polypropylene, polyurethanes, nylon, PTFE and ePTFE.

In another embodiment, at least one nonconductive area comprises a spring hinge panel that has been coated with a dielectric material so as to be electrically insulated from adjacent, conductive spring hinge area. In a still further embodiment, each of the areas may comprise such insulated spring hinge panels. As such, the areas retain their advantageous spring hinge properties, but electricity is prevented from flowing between them even if a portion of the insulation about one or more areas degrades or fails.

### Manufacturing

The deployable electrode patch or net can be formed of a metallic wire, preferably having a shape memory property, covered with a dielectric material. By example, a spring arrays can be formed of drawn Nitinol wire that is coated with silicone. The dielectric coating can also be silicone rubber.

In accordance with one embodiment, the Inventive electrode patch or net is formed into a desired shape before being coated with dielectric material. For example, in one embodiment, Nitinol wire preferably is first treated and shaped to develop a shape memory of a desired spring member structure. Silicone tubing is then pulled over the wire. The wire then is returned to its shape memory shape. In another embodiment, Nitinol wire is dip coated with an insulating material.

It is to be understood that various materials and methods can be used to coat the electrode patch or net with dielectric material. For example, in one embodiment, an etched electrode patch or net is coated with a layer of Parylene™, which is a dielectric polymer available from Union Carbide. Other acceptable materials include silicone rubbers, urethanes, and ceramics, as well as various polymers and the like. The materials can be applied to an etched patch or net construct by various methods, such as dip coating and spraying.

In another embodiment, a portion of the electrode patch or net is electrically insulated by stretching an extruded tube of flexible dielectric material over that area. In a further embodiment, another flexible dielectric tube is disposed on the opposite side of the patch or net to effectively sandwich the patch or net between layers of flexible expandable dielectric material. Gaps may be formed through the dielectric material to help communicate the electric field through the patch or net to the heart.

### Assembly

A connective junction can join various areas of the electrode patch or net. In another embodiment, device parts may be further secured by applying silicone, or another similar material, before the dielectric cover is applied. Also, various portions of the device may be welded, soldered, adhesively bonded, or held together by other means. In still another embodiment, the optional connective junctions may each comprise a small tube segment into which the opposite ends of the device portions are inserted prior to application of the dielectric sheet to the patch or net.

A method of manufacturing the inventive deployable patch or net generally comprises configuring a metallic wire, and then covering the wire with an electrically insulating material. In one embodiment, Nitinol wire is first treated and shaped to develop a “remembered” shape comprising a harness portion and a leader portion of the patch or net. The harness portion is comprised of a plurality of spring members that are preferably arranged into a predefined configuration, such as those shown in the figures. In one embodiment, while held in the predefined configuration, the harness portion is heat-set at a suitable temperature to establish the shape memory. The wire is then electropolished in accordance with standard methods known in the art. The wire is configured such that the leader portion is disposed at one end of the harness portion of the wire.

Once the harness portion of the wire is configured as described above, the wire is then covered with an electrically insulative material. In one embodiment, a tube of dielectric material is pulled over the wire. In certain embodiments, the tube is formed of silicone rubber. It will be appreciated that the inner diameter of the tube determines the level of tightness between the tube and wire. In one embodiment, wherein the wire has a diameter of about 0.012 inches, a silicone tube having an inner diameter of about 0.012 inches provides a relatively tight fit. In another embodiment, wherein the wire has a diameter of about 0.012 inches, a silicone tube having an inner diameter of about 0.020 inches provides a relatively loose fit. A silicone tube having an inner diameter smaller than the diameter of the wire can also be used to obtain a snug fit. In a preferred embodiment, silicone tubing sold under the trademark Nusil MED 4755 is used.

### Delivery

The present inventive electrode patch or net can be deployed in the manner of a heart sock, but in a considerably less invasive manner via a very small subxiphoid or intercostals incision approach. Because of the small profile of the present inventive patch or net, as contrasted with many heart socks, a small fraction of the trauma typically associated with the installment of heart sock is achieved. This provides a much lager group of patients who can benefit from the inventive device. Patients with substantial external cardiac adhesions can be fitted with the device, either by determining a relatively clear area, or by breaking the adhesions in the limited heart surface area to be treated. Additionally, patients with substantially compromised ability to withstand a long or relatively invasive procedure can elect the more limited procedure available with the present invention.

In a standard epicardial device insertion procedure, the inventive device with associated electrodes and leads can be deployed through conventional cardio-thoracic surgical techniques such as through a median sternotomy. In such a procedure, an incision is made in the pericardial sac and the cardiac harness can be advanced over the apex of the heart and along the particularly desired epicardial surface of the heart simply by pushing it on by hand. The intact pericardium is over the patch or net and helps to hold it in place.

The suction grip pads, expandable balloons, and other means previously described to encourage contact of the electrode patch or net device to the surface of the heart, can provide sufficient contact and stabilization of the electrode patch or net to the epicardial surface so that micro-hooks, sutures, clips or staples can be placed during a beating heart procedure. These latter fixation devices could also be bio-absorbable. Other procedures to gain access to the epicardial surface of the heart include making a slit in the pericardium and leaving it open, making a slit and later closing it, or making a small incision in the pericardium.

In certain embodiments, the electrode patch or net associated electrodes and leads may be delivered through minimally invasive surgical access to the heart. A delivery device may be placed intercostal into the thoracic cavity between the patient's ribs to gain direct access to the heart. Typically because of the small profile of the present inventive patch or net, as contrasted with many heart socks and other epicardial devices, this minimally invasive procedure is accomplished on a beating heart, without the use of cardio-pulmonary bypass. Access to the heart can be created with conventional minimally invasive surgical techniques.

For example, a small incision can be made in the pericardium (pericardiotomy) to allow the delivery system access to the heart. The delivery system of the disclosed embodiments comprises several components such as a guidecatheter configured for low profile access using a subxiphoid or intercostal approach. The steerable guidecatheter is inserted into the pericardial cavity and steered to the desired epicardial location of the heart.

In one embodiment the delivered epicardial device has a star configuration with a number of fingers that are flexible and have a small collapsed delivery size, and an expanded size that provides the desired heart surface coverage. Elastic bands can interconnect the distal end of the fingers and prevent the fingers from over-expanding during delivery of the device. The collapsed epicardial device is loaded inside the guidecatheter. Once the guidecatheter is in position, the epicardial device is deployed. The device expands to its shape set configuration as it is advanced out of the distal tip of the guidecatheter into the pericardial cavity.

The device is then fixated to the epicardial surface to ensure good contact between the electrodes or sensors and the heart using one or more of the following techniques: bioabsorbable staples, microhooks, bards, sutures, or adhesive. In another embodiment, a bio-absorbable sock is used to temporarily hold the epicardial device in place until adhesions form.

For larger embodiments of the present epicardial device and in hearts with adhesions, a dilator catheter or RF tunneling device can be used to create an adequate passageway for the delivery guidecatheter.

The delivery system can also include a releasable suction device, such as suction cup at the distal end of a steer-able delivery catheter. The negative pressure suction cup is used to hold the desired portion of the heart. Negative pressure can be applied to the suction cup using a syringe or other vacuum device commonly known in the art. A negative pressure lock can be achieved by a one-way valve stop-cock or a tubing clamp, also known in the art. The suction cup can be formed of a biocompatible material and is preferably stiff enough to prevent any negative pressure loss through the heart while manipulating the heart and sliding the electrode patch or net onto the heart.

Further, the suction cup can be used to lift and maneuver the heart and/or surrounding tissues to facilitate advancement of the electrode patch or net or to allow visualization and surgical manipulation of the posterior side of the heart. The suction cup has enough negative pressure to allow a slight pulling in the proximal direction away from the apex of the heart to somewhat elongate the heart (e.g., into a bullet shape) during delivery to facilitate advancing the patch or net onto the base portion of the heart when placing the patch or net on that area is of clinical value.

After the suction cup is attached to the desired area of the heart and a negative pressure is drawn, the electrode patch or net, which has been releasably mounted in the distal end of the dilator tube, can be advanced distally over the heart.

Visualizing equipment that is commonly known in the art may be used to assist in positioning the delivery guidecatheter and the suction cup to the desired area. For example, fluoroscopy, magnetic resonance imaging (MRI), dye injection to enhance fluoroscopy, and echocardiography, and intracardiac, transesophageal, or transthoracic echo, all can be used to enhance positioning and in attaching the suction cup to the desired region of the heart or positioning the inventive device. After negative pressure is drawn and the suction cup is securely attached (releasably) to the apex of the heart, the heart can then be maneuvered somewhat by pulling on the tubing attached to the suction cup, or by manipulating the introducer tube, the dilator tube, both in conjunction with the suction cup.

Since the electrode patch or net and its attendant devices and electrodes are typically coated with dielectric material, such as silicone rubber, the patch or net will slide easily over the epicardial surface of the heart. The silicone rubber offers little resistance and the epicardial surface of the heart has sufficient fluid to allow the harness to easily slide over the wet surface of the heart.

The pericardium can be cut so that the electrode patch or net slides over the epicardial surface of the heart with the pericardium over the patch or net helping hold it onto the surface of the heart. Prior to removing the introducer tube, a power source (such as an ICD, CRT-D, and/or pacemaker) can be implanted by conventional means. The electrodes will be attached to the pulse generator to provide a defibrillating shock or pacing functions.

Even though the electrodes are designed to be atraumatic and longitudinally flexible, the electrodes have sufficient column strength so that pushing on the proximal ends of the electrodes assists in pushing the patch or net out of the dilator tube and over the epicardial surface of the heart. In one embodiment, advancement of the patch or net is accomplished by hand, by the physician simply pushing on the electrodes and the leads to advance the cardiac harness out of the dilator tube to slide onto the epicardial surface of the heart.

The delivery device typically will have a circular cross-section. It may be desirable, however, to choose other cross-sectional shapes, such as an oval cross-sectional shape for the delivery device. An oval delivery device may be more easily inserted through the intercostal space between the patient's ribs for a low profile delivery. Further, as the patch or net is advanced out of a delivery device having an oval cross-section, the electrode patch or net end will quickly form into a more circular shape in order to assume the configuration of the epicardial surface of the heart as it is advanced distally over the heart.

### Multi-Dimensional Electrode Array Embodiments

These embodiments of the invention are directed to a lead which forms a multi-dimensional electrode array. The lead can be delivered in a straight configuration with the use of guiding catheters or other delivery tools. With the multi-dimensional array lead electrical signals can be generated on any electrodes in the multi-dimensional array. This quality creates unprecedented clinical capabilities to control electrical signals over desired areas of the heart without resorting to surgical repositioning. In the following section, the inventive leads of these embodiments are described primarily in terms of electrode effectors present on the leads. However, it is specifically noted that these embodiments are not so limited, such that the inventive leads may be employed with any type of desirable effector, including any of the effectors described above.

In one embodiment the inventive lead is delivered in a straight configuration with the use of guiding tools. The lead then expands to assume a tortuous configuration when the delivery tools are exited. Several electrodes are disposed along the lead's length. With the lead in a tortuous configuration, the electrodes spread out to form a multi-dimensional electrode array which covers a larger surface area than a straight lead or a single electrode lead.

The multi-dimensional array lead can be easily navigated through tissue, because it will be substantially straight and narrow before the delivery tools are exited. For example, there are often fibrotic adhesions in the space between the epicardium and the pericardial sac, unlike the multi-dimensional array lead, devices that have multiple-fingers or patches will have difficulty navigating this space.

### Shape

In one embodiment, the lead has a sinusoidal shape. This embodiment of the invention may be implanted on a surface, such as the epicardial surface between the epicardium and the pericardial sac to dispose electrodes in an array on the epicardial surface. Further, this embodiment of the invention may be wedged in a vein or between two organs, such as between the epicardium and the pericardial sac, so that it maintains its position against the epicardium by pushing off of the pericardial sac.

In another embodiment, the lead is a sinusoidal shape with decreasing bends in the distal direction. This shape has the advantage of making it easier to exit the delivery tools from the lead. Further, when this embodiment is delivered onto the epicardial surface through the upper portion of the heart, the resulting array of electrodes will cover more surface area in the upper portion of the heart, which is the preferred area for pacing.

Another embodiment of the multi-dimensional array lead is a spiral shaped lead. In this embodiment, electrodes will be disposed on a region inscribed by a circle. This embodiment may be implanted on a tissue surface, such as the epicardial surface between the epicardium and the pericardial sac.

A further embodiment of the multi-dimensional array lead is a lead that turns back on itself to make a “U” shape.

### Electrodes

One embodiment of the multi-dimensional array lead is a lead with conductor cables that run along the length of the lead and connect to a Multiplex chip, which connects to an electrode. In one embodiment of the lead, the electrode and the Multiplex chip may be electrically attached. In another embodiment the Multiplex chip may be electrically attached to the electrode. In a further embodiment the electrode and the Multiplex chip can be built as a monolithic unit. In yet another embodiment, the lead can be made with direct electrical connections to the electrodes, without the Multiplex chip. The electrodes may be made of platinum-iridium and coated with titanium-nitride or iridium-oxide, or any other material suitable for use in a human body.

One embodiment of the multi-dimensional array lead is a lead with electrodes that wrap all the way around the lead body. Another embodiment of this invention is a lead with electrodes exposed on only a partial circumference of the lead, producing a more focused signal. The electrodes may be made of platinum-iridium and coated with titanium-nitride or iridium-oxide, or any other material suitable for use in a human body. With the electrodes exposed on one side, this lead has the ability to pace heart tissue without disturbing other organs, such as the Phrenic nerve.

In one embodiment of the lead with a sinuous shape, the electrodes are disposed at the apex of the bends. This results in the maximal distance between electrodes, and coverage of the largest surface area with electrodes. In another embodiment of the lead with a sinuous shape the electrodes are disposed off of the apex, this configuration has the advantage of making it easier to exit the delivery tools because placing an electrode at the apex may introduce increased bending stiffness coincident with the bends of the lead.

An embodiment of the multi-dimensional array lead may contain pacing electrodes. An embodiment of the multi-dimensional array lead may also contain electrodes for positional measurement with a cardiac resynchronization system, e.g., a SyncAssist□ system (Proteus Biomedical, Redwood City, Calif.). The multi-dimensional array lead provides advantages for use with a cardiac resynchronization system, e.g., a SyncAssist[ ] system (Proteus Biomedical, Redwood City, Calif.) because the multi-dimensional array lead allows for a plurality of positional electrodes to be placed on the epicardial surface to trace the motion of the epicardial surface over a large surface area. Further, the multi-dimensional array lead has an advantage for positional measurement because the placement of electrodes in the multi-dimensional array lead is independent of vein location, thus, with the multi-dimensional array lead positional measurement is not limited by vein location, as it would be with leads that are used strictly in the vein anatomy.

One embodiment of the multi-dimensional array lead is a lead where an electrode is configured so that the anode is placed adjacent to the cathode. In another embodiment an electrode is configured so that the anode is positioned inside of the cathode. Positioning one electrode inside of the other can help focus the signal.

### Tip

An embodiment of the multi-dimensional array lead is a lead with a tip that forms a chisel to allow the lead to be pushed through tissues, such as the adhesions between the epicardial surface and the pericardial sac. Another embodiment of the multi-dimensional array lead is a lead which features a lumen through which a guide wire may be inserted. The lead may be made so that the guide wire will protrude through an opening in the front of the lead body. The lead may also be made so that the guide wire does not penetrate the front of the lead body but rests below the surface. The tip shape may be further modified to provide better dissecting action through tissues such as the adhesions in the interface between the epicardium and the pericardial sac. For example, the tip may be chiseled, blunted, pointed, or have additional radiuses.

### Cross-Sectional Profile

An embodiment of the multi-dimensional array lead is a lead with a circular cross-sectional profile. A lead with such a cross section is ideal in applications where it is desirable to have a lead with similar bending stiffness in all directions.

Another embodiment of the multi-dimensional array lead is a lead with a rectangular cross sectional profile. A lead with such a cross section is ideal in applications where it is desirable to have a lead with different bending stiffness in some directions. A lead with a rectangular cross section, may have advantages for deployment on the epicardial surface, because this lead has less stiffness in the direction perpendicular to the epicardial plane, which allows the lead to bend easily around the epicardial surface and to be easily navigated in the space between the epicardium and the pericardial sac, and because this lead has more stiffness in the direction parallel to the epicardial surface, which allows the lead to maintain its desired shape in the plane of the epicardium. Further, leads could be made with other cross-sectional profiles, such as triangular, square, oval etc.

### Positioning

One embodiment of the multi-dimensional array lead is a lead positioned on the surface of the targeted tissue, such as on the epicardial surface, to create a multi-dimensional electrode array on the surface. Another embodiment of the multi-dimensional array lead is a lead positioned in a plane perpendicular to the targeted tissue, so that the device is wedged in a vein, or between two surfaces such as the epicardium and the pericardial sac. Wedging the lead in a vein, or between two surfaces allows the lead to keep its position against a surface by pushing off of the opposite surface.

One embodiment of the multi-dimensional array lead is a lead that is placed in a vein and then exits the vein where a sealing material seals the vein to prevent leakage. This sealing material may be Dacron fibers, ano-acrylate glues, cellulose glues or other prothrombotic materials to prevent leakage of venus blood into the pericardial space. This procedure may also be done with the use of diarrhetics to control fluid buildup in the space which is entered, such as between the epicardium and pericardial sac following the procedure.

One embodiment of the present invention is a lead which exits a vein at the upper portion of the heart and traverses the space between the epicardial surface and the pericardium. Another embodiment of the multi-dimensional array lead is a sinusoidal shaped lead which enters the surface between the epicardium and the pericardium through a sub-apex approach.

Another embodiment of the multi-dimensional array lead is a lead with a shape that turns back on itself in the upper region of the ventricle, where the lead enters the surface between the epicardium and the pericardium through a sub-apex approach.

To promote adhesion of the lead to organ surfaces, such as the epicardial surface, this lead can be designed with various materials on the outside of the lead body that would increase the thrombotic response. Such materials would include Dacron and other surface chemicals. In addition, surface roughening may be used to promote adhesion of the lead to tissue surface.

### EMBODIMENTS DEPICTED IN FIGURES

Various embodiments of the invention are now reviewed in terms of the figures. FIG. 1A is a depiction of an epicardial multielectrode patch lead 10 employing a multiplexing system present in elongated member 12 and a deployable platform 14 that has pre-shaped spiral configuration which allows it to be deployed minimally invasively via a steerable catheter 16. During deployment, a bioabsorbable clip 17 is used to temporarily fix the epicardial device in place. Deployable patch 14 includes a plurality of effectors, e.g., electrodes, 18. Also shown is an implantable control device 19 which may be an ICD or pacemaker can. FIGS. 1B to 1D are pre-shaped accordion, star or finger configurations of a deployable epicardial platform of alternative embodiments of the invention.

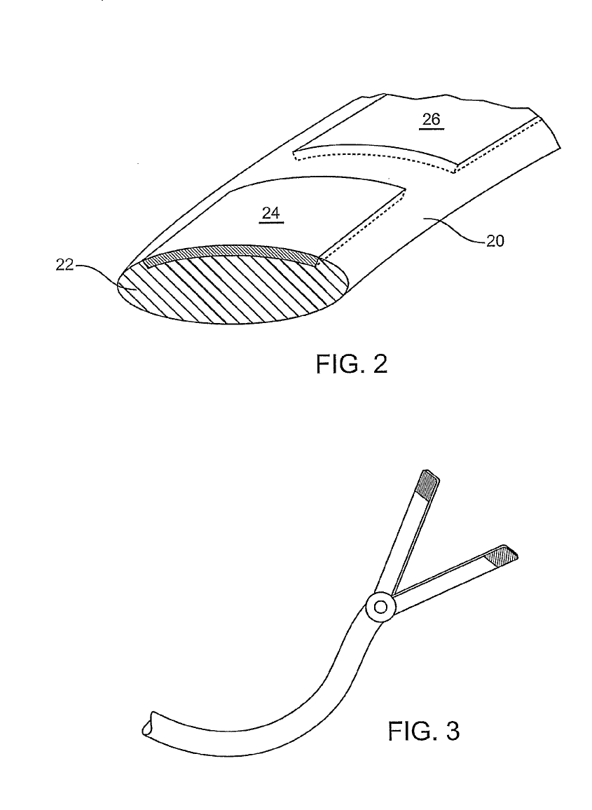

FIG. 2 is partial sectional view of an epicardial multielectrode lead 20 with a flattened cross section 22 and electrodes 24 and 26 exposed only on one side of the lead 20. This design allows the lead to track more easily around the heart with electrodes preferentially oriented to only contact the heart.

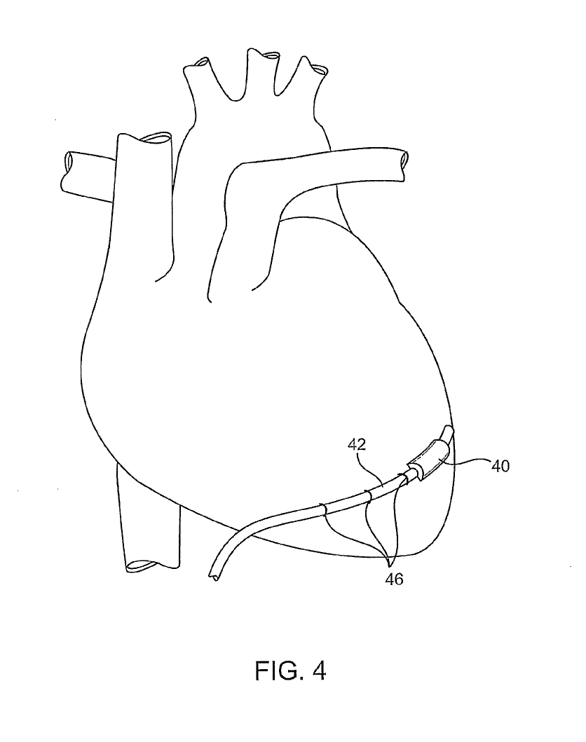

FIG. 3 is a depiction of an RF ablating dissection tool 30 which is used in certain embodiments to tunnel through adhesions in the pericardial space for placement of an epicardial electrode lead of the invention. RF ablation tool has electrodes disposed distally on device. The electrodes are connected to an RF generator thru conductive wires running thru the device. The device dissects tissue thru electrodes disposed on the inside edges of fingers, where the fingers can be pivoted towards each other.

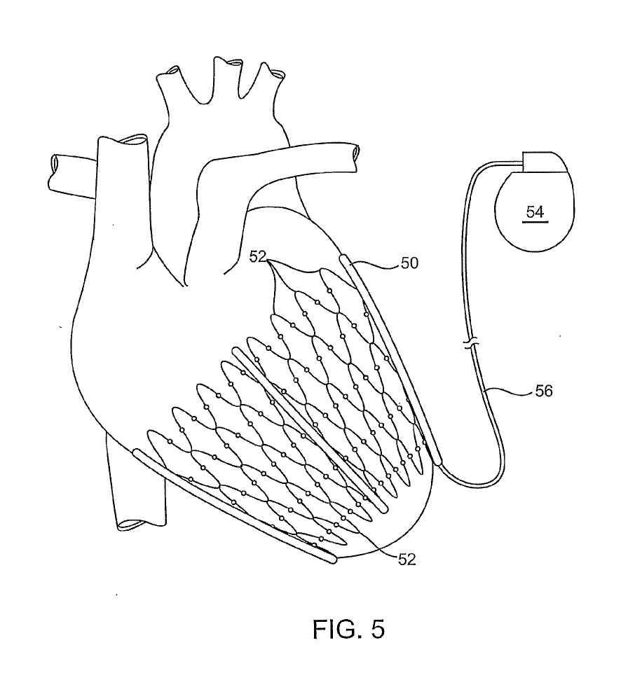

FIG. 4 is a depiction of a steerable rail-guided stapler 40 used for fixation of the epicardial electrode lead 42 to the heart. Shown in FIG. 4 is lead 42 affixed to the epicardial surface by staples 46.

FIG. 5 is a depiction of a multielectrode epicardial heart basket 50. Basket 50 includes a plurality of effectors 51 (e.g., electrodes, sensors, etc) present on a deployable net or mesh support 52 which is configured to cover a region of the epicardial surface of the heart, as shown. The deployable structure 50 is connected to control device 54 by lead 56. The system depicted in FIG. 5 uses one-wire technology and addressable control circuits at each effector as described in published United States Patent Application Publication Number 2006/0058588 to select, pace, and sense from any combination of electrodes of heart basket 50.

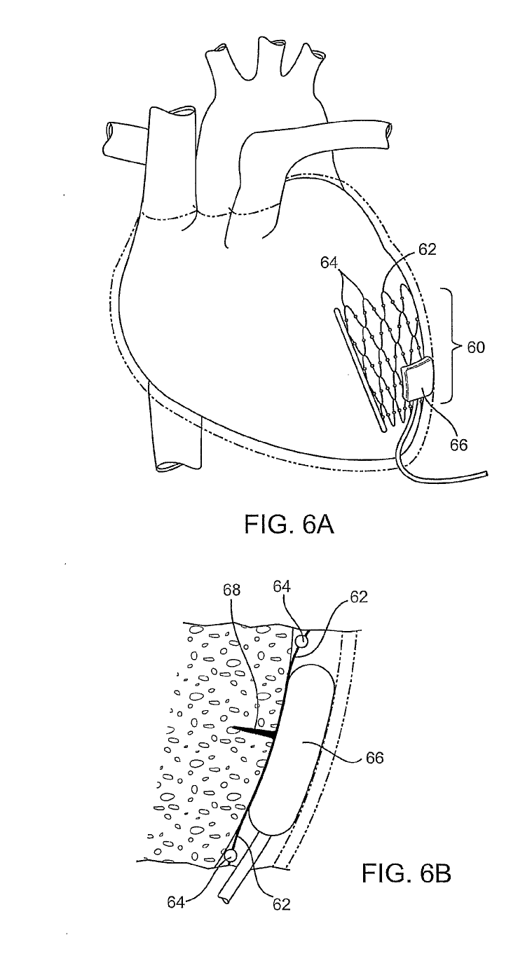

FIG. 6A is a depiction of an epicardial multielectrode expandable net 60 employing a multiplexing system which can be placed via a catheter and then spread-sail deployed at a desired epicardial location. Net 60 includes mesh or net support element 62 and a plurality of effectors 64. Also shown is attachment element 66. FIG. 6B is a depiction of a balloon attachment element 66 that can be used to stabilize the epicardial multielectrode net 60 against the heart during minimally invasive fixation with sutures, staples, or electrically active microhooks, such as element 68.

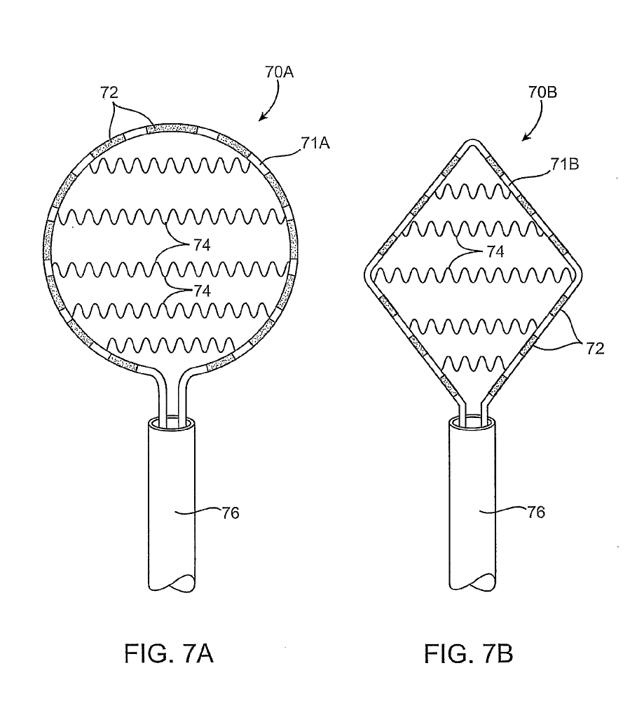

FIG. 7A is a depiction of an epicardial multielectrode net lead 70A employing a pre-shaped loop configuration. Net lead 70A includes ring support 71A on which are positioned a plurality of effectors 72 (electrodes/sensors). Also shown is deployable structural elements 74. The structure is delivered using delivery catheter 76. FIG. 7B is a second embodiment with a pre-shaped rhombus configuration is also shown. Net lead 70B includes ring support 71B on which are positioned a plurality of effectors 72 (electrodes/sensors). Also shown is deployable structural elements 74. The structure is delivered using delivery catheter 76.

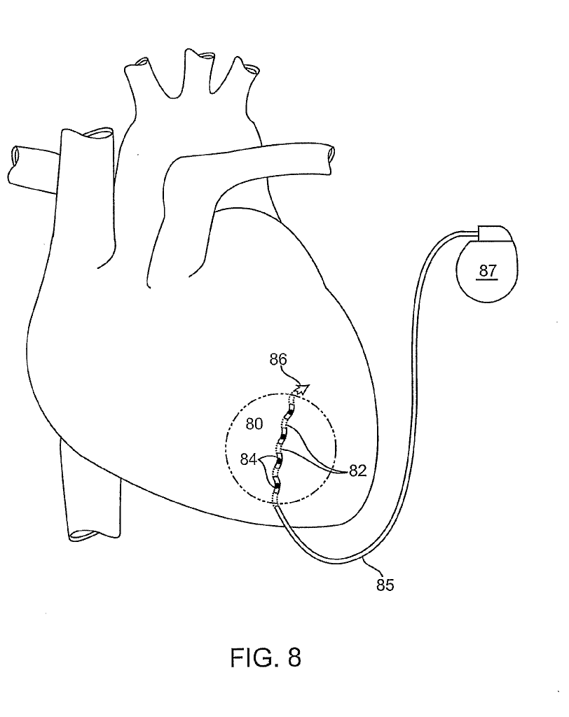

FIG. 8 is a depiction of an epicardial multielectrode lead 80 which is sutured into the heart wall. Lead 80 is sutured to the heart wall by sutures 82 and includes a plurality of effectors 84. Also shown is fixation times 86. The lead is coupled to an implantable control device 87 by elongated member 85.

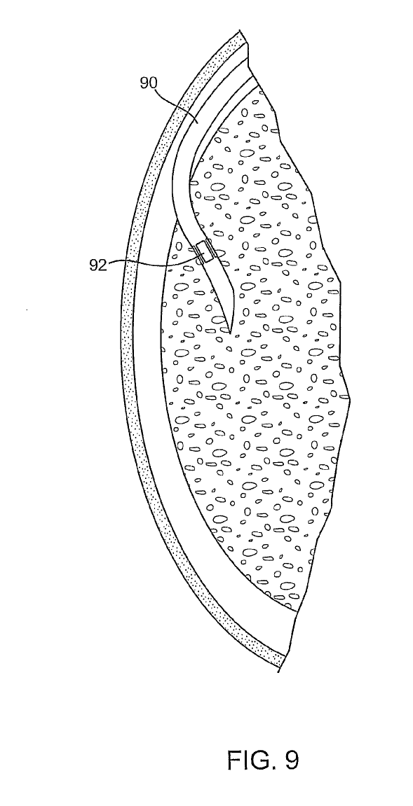

FIG. 9 is a depiction of an epicardial multielectrode lead 90 which is placed in the heart by puncturing the myocardium. A microchip embedded at the electrode location is used to activate any combination of electrodes of the segmented electrode structure 92, e.g., using methods as described above.

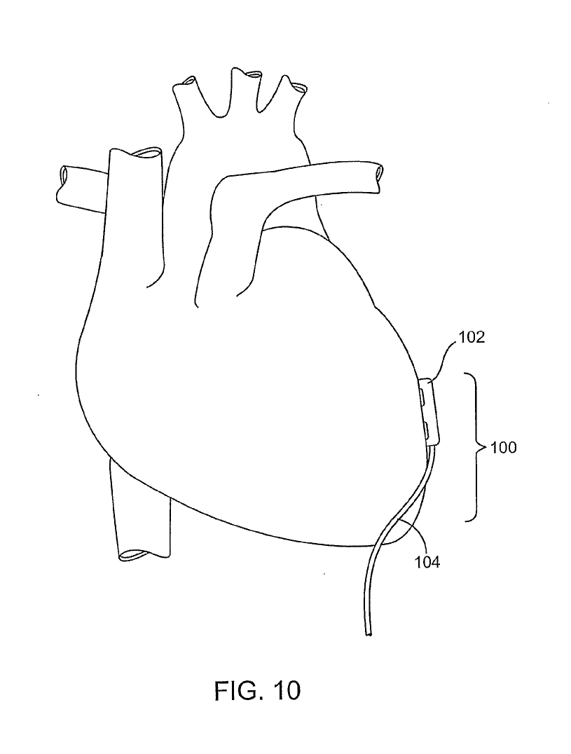

FIG. 10 is a depiction of a steerable flexible suction and delivery device 100 used to stabilize the heart, deliver and attach an epicardial device. Device 100 includes suction element 102 and steerable catheter 104.

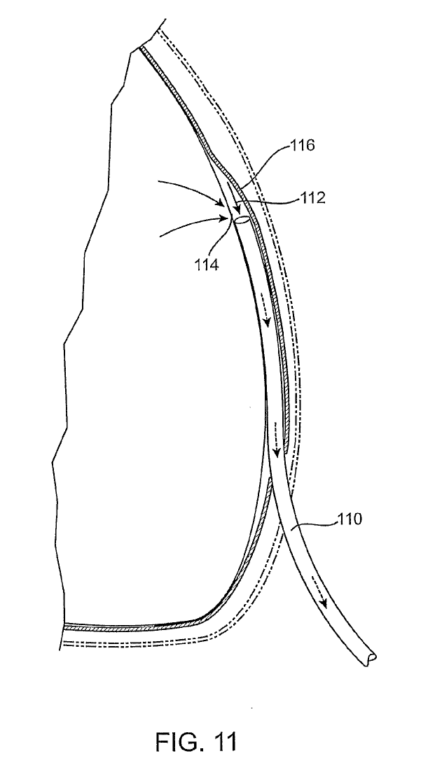

FIG. 11 is a depiction of a suction device 110 used to create a vacuum in the pericardial cavity 112 in order to temporarily stabilize an epicardial device against the heart during fixation. Cavity 112 is bounded by heart wall 114 and pericardium 116 and device 110 removes contents of cavity 112 by sucking in direction of the arrows.

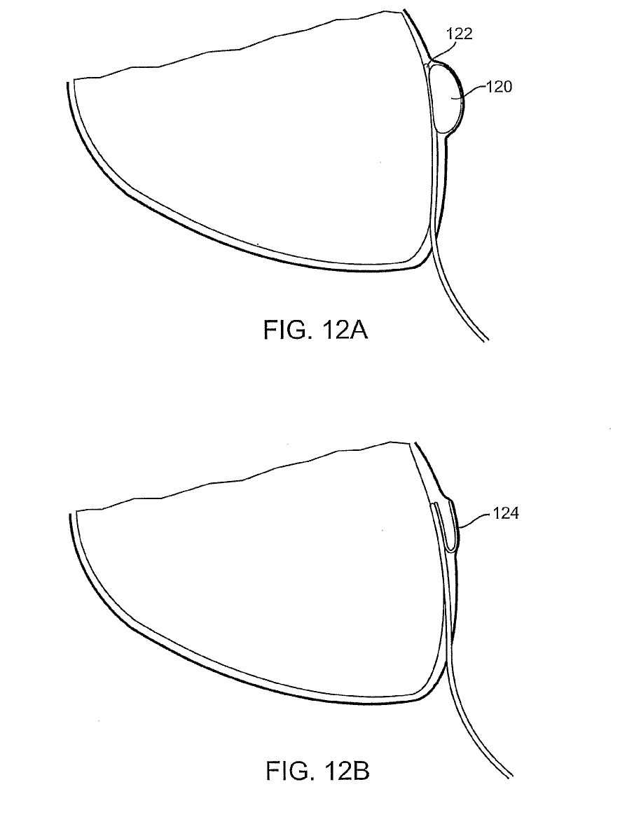

FIG. 12A is a depiction of a balloon device 120 used to temporarily stabilize an epicardial device 122 against the heart during fixation with staples, sutures, or other devices. Also shown in FIG. 12B is a spring clip device 124 with the same purpose.

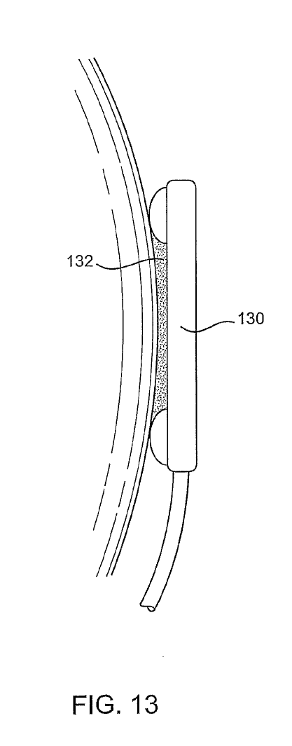

FIG. 13 is a depiction of an attachment element 130 that includes conductive (e.g. Pt microspheres or carbon fibers in cyanoacrylate) or non-conductive adhesive 132 which is used for fixation of the epicardial multielectrode leads.

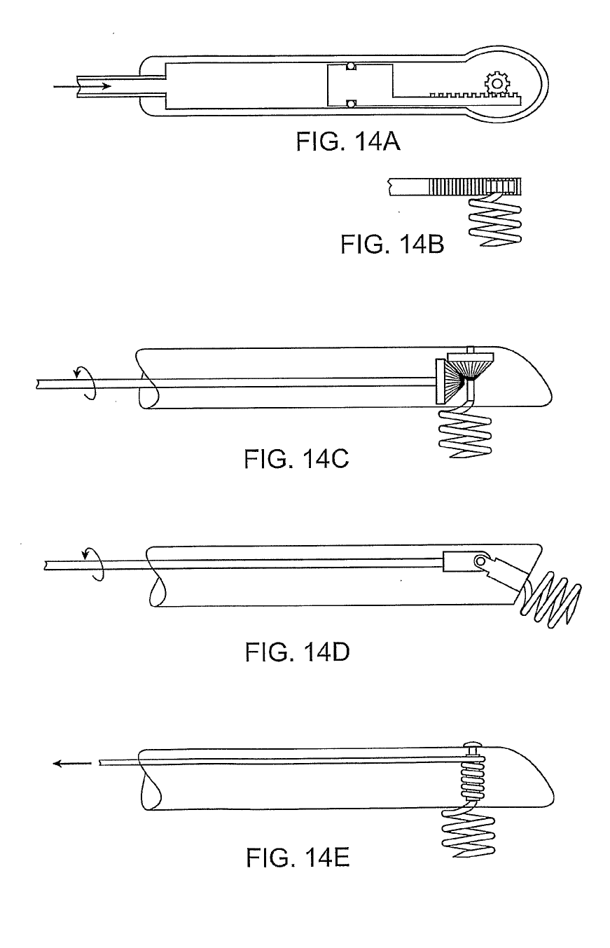

FIGS. 14A to 14E provide depictions of several embodiments of epicardial leads using pneumatic, bevel gears, universal joint, and cable winding mechanisms to rotate the active fixation helix electrode. FIG. 14A depicts a slideable rack gear that engages a rotating pinion gear attached to a helical screw, pacing electrode. The motion of the rack gear drives the helical screw into the tissue. FIG. 14B shows the pinion gear attached to the helical screw, pacing electrode. FIG. 14C shows an conical gear driven by a flexible shaft. The conical gear drives another conical gear that is attached to a helical screw, pacing electrode. The motion of the gear drives the helical screw, pacing electrode into the tissue. FIG. 14D shows an universal joint driven by a flexible shaft. The universal joint is attached to a helical screw, pacing electrode. The motion of the shaft drives the helical screw, pacing electrode into the tissue. FIG. 14E shows an flexible cord that is disposed around the shaft of a helical screw, pacing electrode. The cord is pulled proximally rotating the helical screw, pacing electrode. The rotary motion of the shaft drives the helical screw, pacing electrode into the tissue.

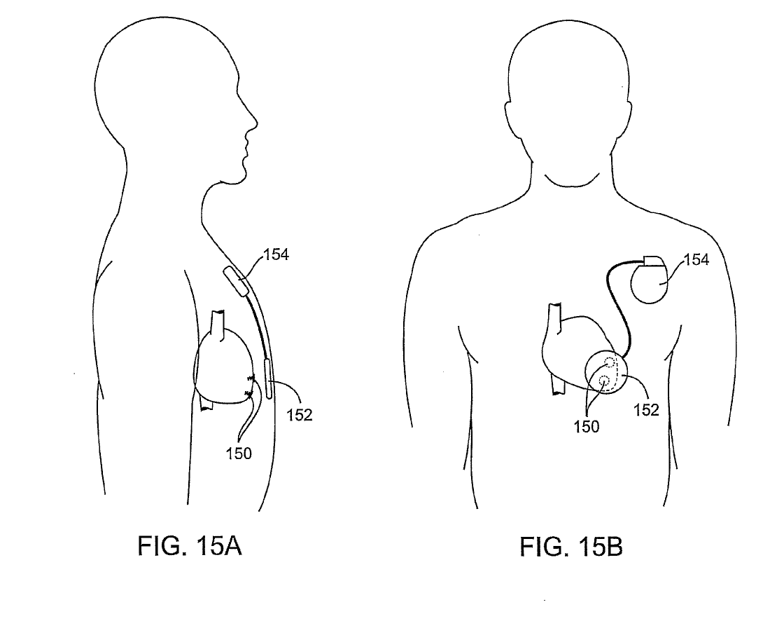

FIGS. 15A and 15B are depictions of wireless epicardial devices 150 which are powered and controlled by a subdermally implanted communication device (e.g. RF coil for power and data transmission) 152 and pacemaker 154. Also of interest for wireless communication are the wireless communication approaches operate at wavelengths much larger than the human body (λ>>1 meter) to communicate information within the patient's body, e.g., as described in U.S. Provisional Application Ser. No. 60,713,680; the disclosure of which is herein incorporated by reference.

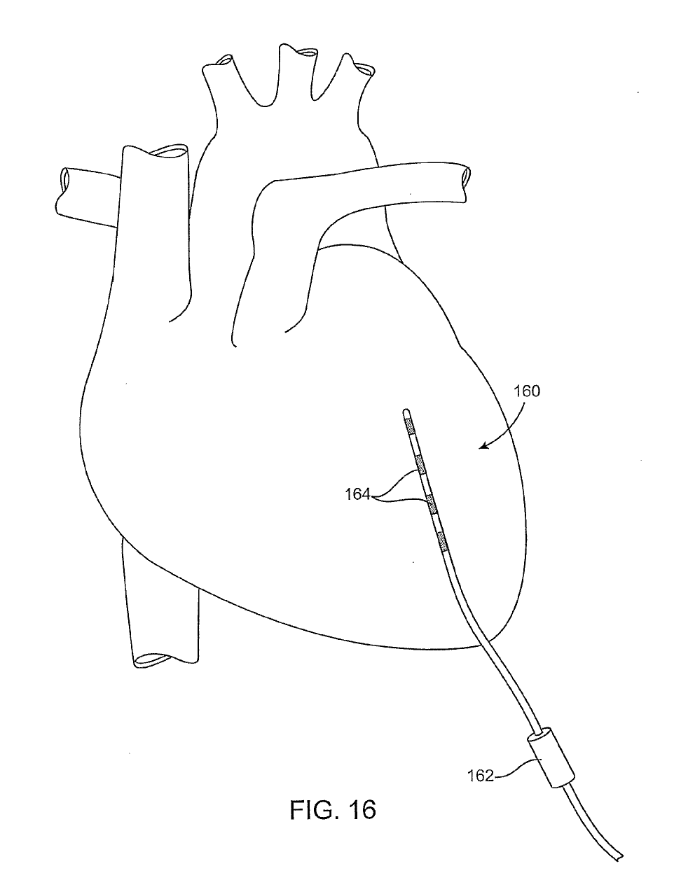

FIG. 16 is a depiction of an epicardial lead that includes a pericardial pressure sensing device 160 which includes various effectors 164 and a differential pressure sensor 162. The pressure sensor monitors fluid pressures in the epicardial space.

FIG. 17A is a depiction of an epicardial partial mechanical constraint device 170 that is comprised of a minimally invasively delivered mesh patch 172 which is locally fixated to the heart using multiple sutures, staples, or microhooks 174. Also shown in FIG. 17B is an epicardial multi-balloon multi-electrode lead 176 employing a pre-shaped spiral element 177 with multiple balloons 178 positioned thereon. A bioabsorbable clip 179 is used to temporarily fix the epicardial device in place. An implantable pacemaker and pump 180 is used to pace and sense from the electrodes 175 and inflate and deflate the balloons for mechanical stimulation.

FIGS. 18 to 25b provide depictions of various embodiments of the Multi-Dimensional electrode lead embodiments of the invention.

FIG. 18 illustrates an embodiment of the multi-dimensional array lead as a sinusoidal shaped lead 3 with uniform bends along the lead's length. Traversing in the distal direction along the lead, a lead body 1 precedes a lead body which is contoured into a tortuous configuration 3. A plurality of electrodes 5 is disposed along the lead body 3. The lead is delivered in a straight configuration with the use of guiding catheters or other delivery tools. The lead expands to assume its tortuous configuration when the delivery tools are exited. With the lead in a tortuous configuration, the electrodes spread out to form a multi-dimensional array which covers a larger surface area than a straight lead or a single electrode lead.

The lead may be deployed on the epicardial surface in the space between the pericardial sac and the epicardium. The lead may also be deployed on other tissue interfaces such as the diaphragm or other organs in the heart. Further, the lead may be positioned so that the lead's tortuous shape allows it to be wedged between tissues, such as in a vein or artery. The lead may also be positioned so that it is wedged between the epicardium and the pericardial sac.

To promote adhesion of the lead to organ surfaces, such as the epicardial surface, this lead can be designed with various materials on the outside of the lead body that would increase the thrombotic response. Such materials would include Dacron and other surface chemicals. In addition, surface roughening may be used to promote adhesion of the lead to tissue surface.

FIG. 19a-19c illustrate shape factors of various embodiments of the invention. FIG. 19a illustrates an embodiment of the invention as a sinusoidal shaped lead 8 with decreasing bends 9 in the distal direction, and electrodes 7 located at the apex of the bends of the lead. As illustrated in FIG. 25a, when a lead 10 with this shape factor enters the epicardial surface from the top of the heart, it will cover a larger surface area in the upper portion of the heart than at the bottom. Typically, the area of interest for pacing the left ventricle is in the upper third of the outside of the ventricle. It is advantageous to have a lead shape that covers the maximal surface area in the upper portion of the ventricle, and less surface area as the lead traverses away from this region.

Further, covering a large surface area of a ventricle lowers the probability that electrodes 7 are located over locations of infarcts. Also, decreasing bends in the distal direction of the lead will allow easier exiting of the delivery tools. Lastly, by disposing electrodes 7 along the apexes of the bends, this design produces maximal spacing between electrodes 7 and delivers electrical signals on the maximal surface area.

FIG. 19b illustrates an embodiment of the multi-dimensional array lead as a sinusoidal shaped lead 10 with uniform bends along its length, and electrodes 7 located off of the apex of the bends to accommodate mechanical flexures and prevent sticking as the lead exits from the delivery tools. The electrodes on a lead may provide a hard part which will resist bending. Locating the electrodes on the apex may create a discontinuity in bending stiffness coincident with the apex, which may result in locking as the delivery tools exit the lead. The electrodes in this lead are disposed off of the apex to help avoid this problem.

FIG. 19c illustrates a further embodiment of the multi-dimensional array lead as a spiral shaped lead 6. This configuration creates a distribution of electrodes 7 over a circular region and can be deployed over tissue surfaces such as the epicardial surface.

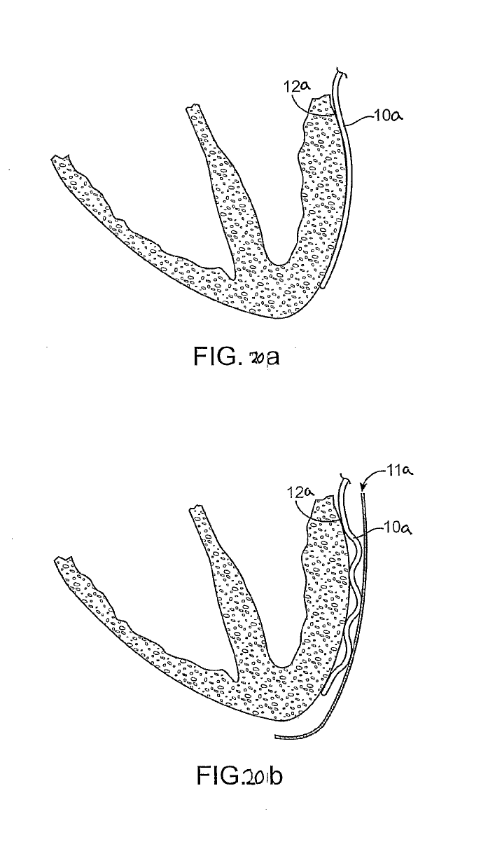

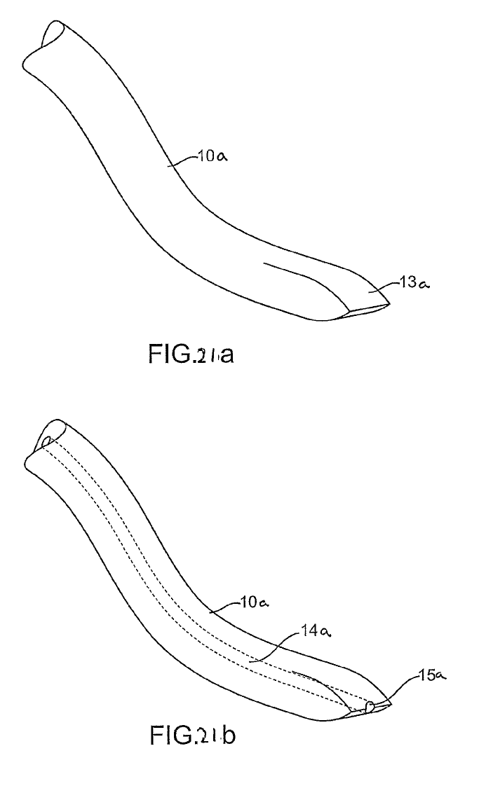

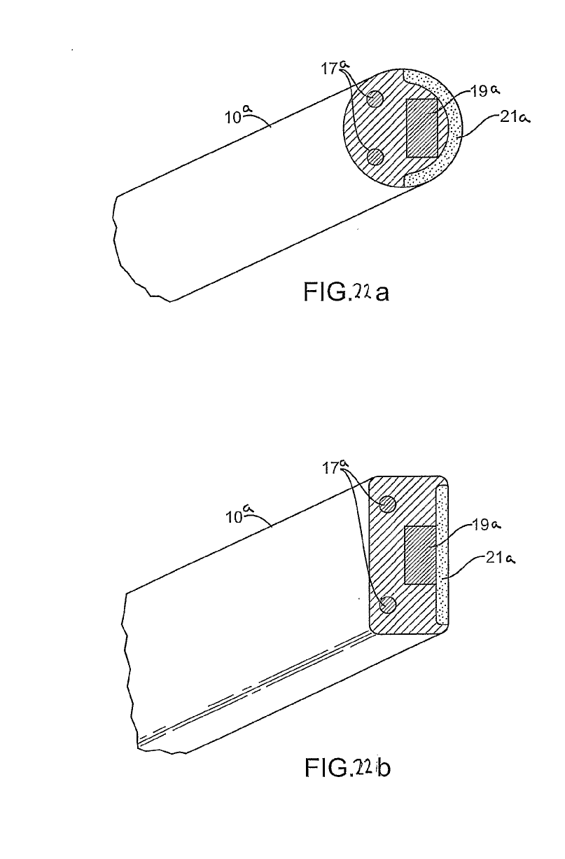

FIG. 20a is an illustration of the multi-dimensional array lead with the lead 10a positioned in the plane of the epicardium 12a surface. FIG. 20b is an illustration of the multi-dimensional array lead with the lead 10a positioned in a plane perpendicular to the epicardium 12a surface, so that the device is wedged between the epicardium 12a and the pericardial sac 11a. Wedging the lead between the epicardium 12a and the pericardial sac 11a allows the lead to keep its position against the epicardium 12a by pushing off of the pericardial sac 11a.

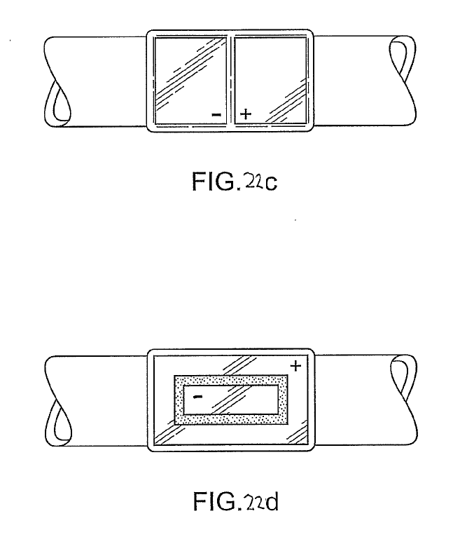

FIG. 21a illustrates an embodiment of the multi-dimensional array lead where the tip 13a of the lead 10a forms a chisel to allow the lead to be pushed through tissues, such as the adhesions between the epicardial surface and the pericardial sac. FIG. 21b illustrates an embodiment of the multi-dimensional array lead where the lead 10a features a lumen 14a through which a guide wire may be inserted. The lead 10a may be made so that the guide wire will protrude through an opening in the front of the lead body 15a. The lead 10a may also be made so that the guide wire does not penetrate the front of the lead body but rests below the surface. The tip shape may be further modified to provide better dissecting action through tissues such as the adhesions in the interface between the epicardium and the pericardial sac. For example, the tip may be chiseled, blunted, pointed, or have additional radiuses.

One embodiment of the multi-dimensional array lead is a lead with electrodes that wrap all the way around the lead body. Another embodiment of the multi-dimensional array lead is a lead with electrodes exposed on only a partial circumference of the lead. FIGS. 22a and 22b are cross-sectional views of a lead 10a with electrodes 21a exposed on one side of the lead, conductor cables 17a, and a Multiplex chip 19a. The electrode 21a and the Multiplex chip 19a in this lead 10a may be electrically attached or connected, or the electrode 21a and the Multiplex chip 19a could be built as a monolithic unit. In certain embodiments, the electrode is present as a segmented electrode structure, e.g., as described above. The lead 10a could be made with direct electrical connections to the electrodes 21a, without the Multiplex chip 19a. The electrodes 21a may be made of platinum-iridium and coated with titanium-nitride or iridium-oxide, or any other material suitable for use in a human body. With the electrodes 21a exposed on one side, this lead 10a has the ability to pace heart tissue without disturbing other organs, such as the Phrenic nerve.

FIG. 22a is a cross-section profile of an embodiment of the multi-dimensional array lead as a lead 10a with a circular cross-section. A lead with such a cross section is ideal in applications where it is desirable to have a lead with similar bending stiffness in all directions. FIG. 22b is a cross-section profile of an embodiment of the multi-dimensional array lead as a lead 10a with a rectangular cross section. A lead with such a cross section is ideal in applications where it is desirable to have a lead with different bending stiffness in some directions.

A lead with a rectangular cross section, such as FIG. 22b, may have advantages for deployment on the epicardial surface, because this lead has less stiffness in the direction perpendicular to the epicardial plane, which allows the lead to bend easily around the epicardial surface and to be easily navigated in the space between the epicardium and the pericardial sac, and because this lead has more stiffness in the direction parallel to the epicardial surface, which allows the lead to maintain its natural shape in the plane of the epicardium. Further, leads could be made with other cross-sectional profiles, such as triangular, square, oval etc.

FIG. 22c is an embodiment of the multi-dimensional array lead where an electrode is configured so that the anode is placed adjacent to the cathode. FIG. 22d is an embodiment of the multi-dimensional array lead with an electrode configured so that the anode is positioned inside of the cathode. Positioning one electrode inside of the other can help focus the signal.

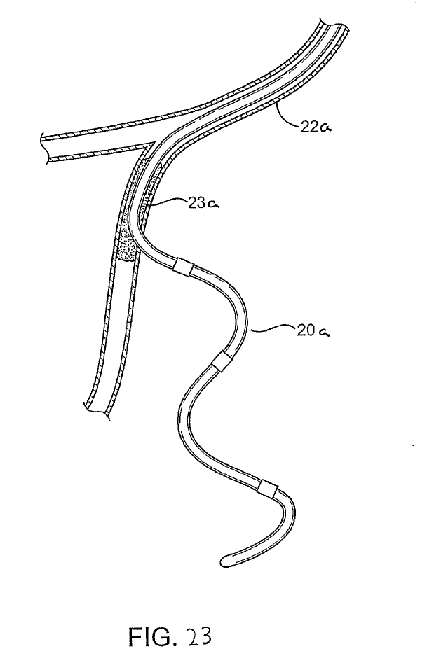

FIG. 23 is an illustration of an embodiment of the present invention as a lead 20a that is placed in a vein 22a and then exits the vein where a sealing material 23a seals the vein to prevent leakage. This sealing material may be Dacron fibers, ano-acrylate glues, cellulose glues or other prothrombotic materials to prevent leakage of venus blood into the pericardial space. This procedure may also be done with the use of diarrhetics to control fluid buildup in the space between the epicardium and pericardial sac following the procedure.

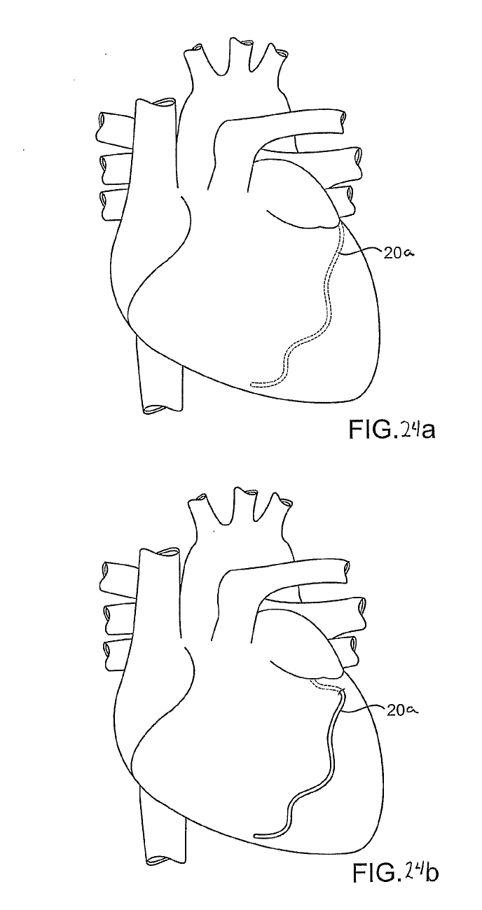

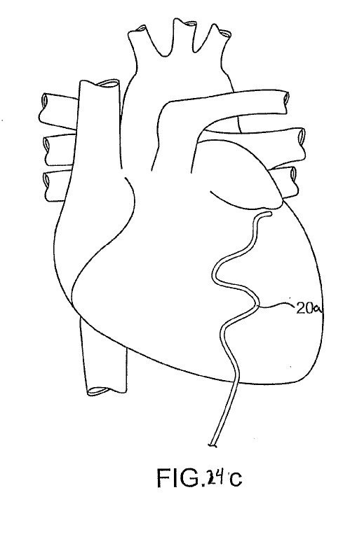

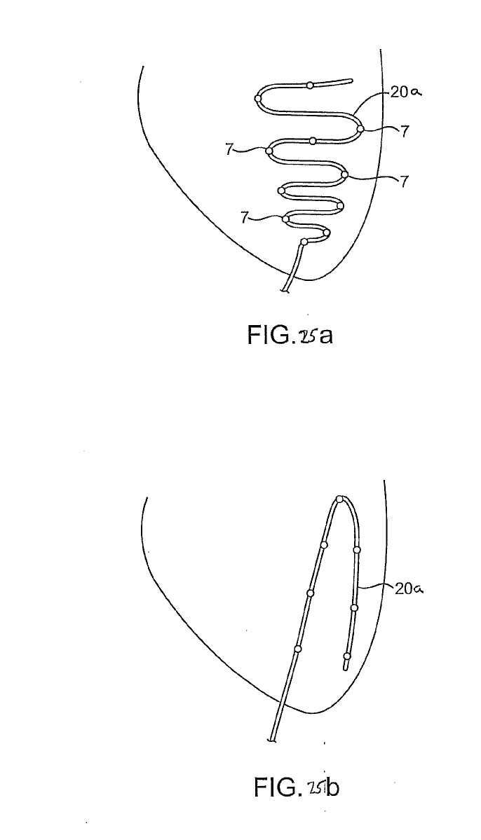

FIG. 24a is an illustration of an embodiment of the present invention as a sinusoidal shaped lead 20a placed in a vein on the outside of the heart. FIG. 24b is an illustration of an embodiment of the present invention as a sinusoidal shaped lead 20a which exits a vein and traverses the space between the epicardial surface and the pericardium. FIG. 24c is an illustration of an embodiment of the present invention as a sinusoidal shaped lead 20a which enters the surface between the epicardium and the pericardium through a sub-apex approach.

FIG. 25a is an illustration of an embodiment of the multi-dimensional array lead as a sinusoidal shaped lead 20a having a plurality of electrodes 7 present thereon, where the lead enters the surface between the epicardium and the pericardium through a sub-apex approach. FIG. 25b is an illustration of an embodiment of the multi-dimensional array lead as a “U” shaped lead 20a that turns back on itself in the upper region of the ventricle, where the lead enters the surface between the epicardium and the pericardium through a sub-apex approach.

### Systems

Aspects of the invention include systems, including implantable medical devices and systems, which include the devices of the invention. The systems may perform a number of different functions, including but not limited to electrical stimulation applications, e.g., for medical purposes, such as pacing, CRT, etc.

The systems may have a number of different components or elements in addition to the epicardial arrays, where such elements may include, but are not limited to: sensors (e.g., cardiac wall movement sensors, such as wall movement timing sensors); processing elements, e.g., for controlling timing of cardiac stimulation, e.g., in response to a signal from one or more sensors; telemetric transmitters, e.g., for telemetrically exchanging information between the implantable medical device and a location outside the body; drug delivery elements, etc. As such, the subject arrays may be operably coupled, e.g., in electrical communication with, components of a number of different types of implantable medical systems, where such systems include, but are not limited to: physiological parameter sensing devices; electrical (e.g., cardiac) stimulation devices, etc.

In certain embodiments of the subject systems, one or more deployable epicardial arrays of the invention are electrically coupled to at least one elongated conductive member, e.g., an elongated conductive member present in a lead, such as a cardiovascular lead. In certain embodiments, the elongated conductive member is part of a multiplex lead, e.g., as described in Published PCT Application No. WO 2004/052182 and U.S. patent application Ser. No. 10/734,490, the disclosure of which is herein incorporated by reference. In some embodiments of the invention, the devices and systems may include onboard logic circuitry Pr a processor, e.g., present in a central control unit, such as a pacemaker can. In these embodiments, the central control unit may be electrically coupled to one or more deployable arrays via one or more conductive members.

In certain embodiments, the implantable medical systems which include the subject deployable epicardial are ones that are employed for cardiovascular applications, e.g., pacing applications, cardiac resynchronization therapy applications, etc.

### Kits

Also provided are kits that include the subject deployable epicardial arrays, as part of one or more components of an implantable device or system, such as the devices and systems reviewed above. In certain embodiments, the kits further include at least a control unit, e.g., in the form of an ICD or pacemaker can. In certain of these embodiments, the structure and control unit may be electrically coupled by an elongated conductive member. In certain embodiments, the kits may further include a delivery device, e.g., a steerable catheter. In certain embodiments, the kits may include a tissue separator, e.g., as shown in FIG. 3. In certain embodiments, the kits may include one or more attachment elements, e.g., as described above.

In certain embodiments of the subject kits, the kits will further include instructions for using the subject devices or elements for obtaining the same (e.g., a website URL directing the user to a webpage which provides the instructions), where these instructions are typically printed on a substrate, which substrate may be one or more of: a package insert, the packaging, reagent containers and the like. In the subject kits, the one or more components are present in the same or different containers, as may be convenient or desirable.

It is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior-invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A minimally invasive deployable epicardial array device comprising:
a deployable platform comprising two or more effectors;
wherein said platform is configured to be deployed at an epicardial location via a sub-xiphoid approach.

2. The minimally invasive deployable epicardial array device according to claim 1, wherein said two or more effectors are actuators or sensors.

3. The minimally invasive deployable epicardial array device according to claim 2, wherein at least one of said effectors is an electrode.

4. The minimally invasive deployable epicardial array device according to claim 3, wherein said electrode is a segmented electrode.

5. The minimally invasive deployable epicardial array device according to claim 1, wherein at least one of said effectors is a mechanical stimulator.

6. The minimally invasive deployable epicardial array device according to claim 5, wherein said mechanical stimulator is a balloon.

7. The minimally invasive deployable epicardial array device according to claim 1, wherein said deployable platform is a net.

8. The minimally invasive deployable epicardial array device according to claim 1, wherein said device includes an attachment element.

9. The minimally invasive deployable epicardial array device according to claim 8, wherein said attachment element is a suction element.

10. The minimally invasive deployable epicardial array device according to claim 8, wherein said attachment element is a permanent attachment element.

11. The minimally invasive deployable epicardial array device according to claim 1, wherein said platform comprises a shape memory material.

12. The minimally invasive deployable epicardial array device according to claim 1, wherein said two or more effectors are conductively coupled to a multiplex lead.

13. The minimally invasive deployable epicardial array device according to claim 12, wherein said multiplex lead comprises one wire.

14. The minimally invasive deployable epicardial array device according to claim 1, wherein at least one of said effectors comprises a processor.

15. The minimally invasive deployable epicardial array device according to claim 14, wherein said processor is a control circuit having a miniaturized form factor.

16. The minimally invasive deployable epicardial array device according to claim 1, wherein said device comprises an elongate member having a distal end and a proximal end, and said deployable platform is positioned at said distal end.

17. The minimally invasive deployable epicardial array device according to claim 16, wherein said proximal end is coupled to an implantable control device.

18. The minimally invasive deployable epicardial array device according to claim 17, wherein said device is configured to wirelessly communicate with a distinct receiver device.

19. The minimally invasive deployable epicardial array device according to claim 17, wherein said distinct receive device is an implanted device.

20. The minimally invasive deployable epicardial array device according to claim 17, wherein said distinct receive device is an ex vivo device.

21. The minimally invasive deployable epicardial array device according to claim 1, wherein said device is associated with a delivery element.

22. The minimally invasive deployable epicardial array device according to claim 21, wherein said delivery element is a catheter.

23-32. (canceled)

33. A system comprising:
a deployable platform comprising two or more effectors; and
a control unit communicably associated with the deployable platform,
wherein the deployable platform is configured to be deployed at an epicardial location via a sub-xiphoid approach.

34. The system according to claim 33, wherein said deployable platform and control unit are electrically coupled by at least one elongated conductive member.

35. The system according to claim 34, wherein said elongated conductive member comprises a multiplex lead.

36. The system according to claim 35, wherein said multiplex lead is a one-wire multiplex lead.

37. The system according to claim 33, wherein said device and control unit are configured for wireless communication with each other.

38. The system according to claim 33, wherein said control unit is present in an implantable control device.

39. The system according to claim 38, wherein said implantable control device is a pacemaker can.

40-49. (canceled)
